(19) United States
(12) Patent Application Publication
(10) Pub. No.: US 2010/0062536 A1
(43) Pub. Date: Mar. 11, 2010

(54) DETECTOR FOR THE DETECTION OF CHEMICAL WARFARE AGENTS AND METHOD OF MANUFACTURE THEREOF

(75) Inventors: Martin Eickhoff, Giessen-Allendorf (DE); Andreas Helwig, Muenchen (DE); José Antonio Garrido, Erding (DE); Gerhard Mueller, Grafing (DE)

Correspondence Address: CROWELL & MORING LLP;INTELLECTUAL PROPERTY GROUP, P.O. BOX 14300, WASHINGTON, DC, 20044-4300, US

(73) Assignee: EADS Deutschland GmbH

(21) Appl. No.: 12/514,739

(22) PCT Filed: Nov. 12, 2007
(86) PCT No.: PCT/DE07/02040

§ 371 Date: Nov. 13, 2009

(30) Foreign Application Priority Data
Nov. 14, 2006 (DE) 10 2006 053 890.0

(51) Int. Cl. G01N 33/20; G01N 27/12; G01N 33/22; H01R 43/00

(52) U.S. Cl. 436/73; 422/98; 436/104; 29/825

(57) ABSTRACT

In a method and apparatus for detecting toxic chemical hazardous materials and warfare agents in a gas, the gas is exposed to a substrate with a hydrogen terminated surface. The substrate may be made from a nonconductor material with available surface conductivity, or from a semiconductor material. The electrical resistance at the hydrogen terminated surface is measured to detect the presence of the hazardous materials and warfare agents. A method of producing such a substrate detector is also disclosed.

This application is a national stage of PCT International Application No. PCT/DE2007/002040, filed Nov. 12, 2007, which claims priority under 35 U.S.C. §119 to German Patent Application No. 10 2006 053 890.0, filed Nov. 14, 2006, the entire disclosure of which is herein expressly incorporated by reference.

### BACKGROUND AND SUMMARY OF THE INVENTION

The invention relates to a warfare agent detector for detection of chemical warfare agents in a gas, and to a process for producing such a detector.

Chemical warfare agents are substances which give rise to acute and chronic health risks. They are highly toxic to humans and animals and extremely environmentally damaging. Examples include phosphine (PH₃), arsine (AsH₃) and diborane (B₂H₆). These also form, for example, a group of highly toxic dopants which are also used routinely in the semiconductor industry. A further group relates to chemical warfare agents which were used as weapons of mass destruction in many countries at the command of the particular decision makers in past wars. Representatives of this group are mustard gases, organophosphorus substances, phosgene, VX, sarin, tabun, and soman. Present-day endangerment by such substances is conceivable as a consequence of accidents with legacy materials or as a result of terrorist attacks.

There is therefore a great need to be able to rapidly detect the presence of such highly dangerous substances in various environments, especially in the air.

Several sensor systems for detection of the abovementioned gases and vapors are on the market. Some of these are expensive and highly complex analytical instruments which can be operated only by trained personnel and whose results can be interpreted reliably only by such personnel. Examples are gas chromatographs, mass spectrometers and ion mobility spectrometers. Sensors available inexpensively on the market, for example thick layer metal oxide sensors, can detect such hazardous gases in low concentrations. However, a serious disadvantage of such inexpensive sensors is the fact that they also react to a large number of disruptive gases which may be present in the air in relatively high and highly variable concentrations. One reason for the inadequate selectivity is the high operating temperature of approx. 400° C., at which most molecules to be detected are burnt at the sensor surface, and are only detected electrically as a result. For more exact identification, it is therefore necessary to operate multiple sensors with different cross-sensitivities in parallel in a sensor array (so-called electronic noses). However, a clear detection of chemical warfare agents with the aid of electronic noses has to date not yet been possible with the necessary reliability in most cases, for the reasons mentioned above.

It is therefore an object of the present invention to enable rapid and uncomplicated detection of chemical warfare agents in the air at room temperature, without reacting to the disruptive gases which likewise occur in various industrial environments and/or in the outdoor environment.

This and other objects and advantages are achieved by the warfare detection process according to the invention, for detecting chemical warfare agents in a gas, and by the warfare agent detector, which comprises a substrate with a hydrogen (H)-terminated surface. The substrate is manufactured from a nonconductor material with an available surface conductivity or from a semiconductor material.

The inventive detection technique makes it possible to detect chemical warfare agents in gases and especially in the air, without any disruption to the warfare agent detection by other substances that may be present. This means that masking of the measured signal caused by the highly toxic or deadly hazardous materials or warfare agents by signals caused by troublesome background substances is prevented. Such disruptive gases are, for example, CO, O3, natural gas (especially CH4) or else alcoholic vapors.

Advantageously, the density of the hydrogen coverage on the surface is reduced by partial oxidation, which enhances the sensitivity of the detection.

According to the invention, the substrate is suitable especially for detection of highly toxic hazardous materials or warfare agents which, in chemical terms, belong to the groups of the III-H, V-H or VI-H compounds, and for detection of molecules which have such reactive groups as molecule constituents.

The invention is based on the consideration that most of these substances have electron orbitals which are doubly occupied and are not involved in any covalent bond within the molecule to be detected. The hydrogenated or hydrogen-terminated surface of the substrate offers a means of docking via hydrogen bonds. The measured results described below demonstrate that electrical charges can be transferred between the warfare agent compounds and the sensor substrate with sufficiently high probability via such physisorption bonds. Such charge transfers lead to a change in the electrical conductivity at the substrate surface and hence to a detectable sensor signal.

Displacements of surface charges are preferably measured at the hydrogen (H)-terminated surface by means of a measuring device.

More particularly, a layer of hydrogen atoms applied to the substrate constitutes the hydrogen (H)-terminated surface. The layer of hydrogen atoms is, for example, a monoatomic layer.

The substrate may be manufactured, for example, from diamond, hydrogenated amorphous silicon (a-Si:H), silicon carbide, a group III nitride or a metal oxide. Specific examples from the latter two groups are GaN or tin oxide, or else zinc oxide.

The electrical resistance is preferably measured on the hydrogen (H)-terminated surface by means of a measuring device. The measurement for detection of the gases or vapors specified is effected preferably at a temperature below 100° C., especially at room temperature.

The sensitive hydrogen (H)-terminated substrate surface is preferably cleaned by purging with a fluid which does not contain the substances to be detected, especially air. The cleaning is preferably effected with addition of an oxidizing fluid, especially ozone.

According to the invention, the substrate is used especially for detection of one or more of the following gases: mustard gas, sulfur mustard gas, organophosphorus warfare agents, nitrogen oxides, B₂H₆, PH₃, AsH₃ or other gases containing group III elements, especially Al(CH₃)₃, Ga(CH₃)₃, In(CH₃)₃.

The inventive warfare agent detector is suitable for detection of chemical warfare agents in a gas and comprises a substrate which has a hydrogen (H)-terminated substrate surface for exposure to the gas, and a measuring device for measuring displacements of surface charges at the hydrogen (H)-terminated substrate surface. The substrate being manufactured from a nonconductor material with surface conductivity or from a semiconductor material.

To form the hydrogen (H)-terminated substrate surface, a layer of hydrogen atoms is preferably applied to the substrate. Due to the hydrogenated surface, the warfare agent detector according to the invention is especially suitable for detection of highly toxic hazardous material or warfare agent compounds of at least one of group III, group V or group VI elements.

A readout unit may be present, for example, in the form of a transistor. The readout unit and the sensitive layer, for example a-Si:H or H-terminated diamond, may be manufactured from different materials. That is, the sensitive layer is first applied to the readout unit.

According to the invention, a semiconductor surface, for example, is terminated with a layer of atomic hydrogen. Such a hydrogenated semiconductor surface constitutes the sensitive layer onto which the warfare agents to be detected dock. The H termination enables, for example, addition of a group V-H molecule to be detected, and the subsequent formation of an electrically charged complex of this molecule with the surface-bonded hydrogen at room temperature. Disruptive gases react at the surface only at relatively high temperatures and thus do not constitute any risk of distortion.

The invention thus enables selective identification of chemical warfare agents in the form of group III-H, group V-H and group VI-H gases and vapors at room temperature.

The hazardous material and warfare agent detector according to the invention has a lower power consumption than existing competing products, since no heating power is required for the sensitive layer. Moreover, the facts that only a single sensor is sufficient, and that no further sensors are required to filter out disruptive gases, facilitate an uncomplicated construction, and further lower power consumption.

Furthermore, the inventive sensor is notable in that it has no cross-sensitivity to environmental influences and disruptive gases in industrial manufacture. After one measurement, the sensor surface can be cleaned by a high concentration of ozone.

The operational principle of the invention (that various group V-H compounds cause a change in the surface conductivity of hydrogenated semiconductor surfaces) has long been known in the literature, at least in the case of hydrogenated amorphous silicon. This material is being used to an enhanced degree in photovoltaics.

For instance, the article “Effect of Adsorbates and Insulating Layers on the Conductance of Plasma Deposited a-Si:H*” by M. Tanielian et al. in Journal of Non-Crystalline Solids 35 & 36 (1980) 575-580 states that various adsorbed gases such as H₂O and particularly NH₃ have a strong effect on the conductivity and the photoconductivity of high-resistance layers of amorphous hydrogenated silicon. It is stated that NH₃ acts as a surface donor for hydrogenated amorphous silicon and thus changes the surface conductivity. In contrast, selenium acts as an acceptor and likewise changes the surface conductivity. It is further stated that changes in surface charge caused by these substances can be reversed by heating under reduced pressure.

In “Chemical Reactions in Plasma Deposition”, chapter 8 from “Semiconductors and Semimetals”, Vol. 21A, Academic Press, Inc., 1984, Frank J. Kampas describes which chemical processes proceed in the growth of hydrogenated amorphous silicon layers and how a naturally hydrogenated surface forms due to the specific growth process. In the article “Hydrogen Incorporation, Doping and Thickness Dependent Conductivity in Glow Discharge Deposited a-Si:H Films”, (Journal of Non-Crystalline Solids 59 & 60 (1983), p. 469-472) G. Müller et al present hydrogen depth profiles through a-Si:H layers of various thickness and show explicitly that a high surface density of hydrogen atoms is present, which is consistent with the forecasts of Kampas and Griffith.

In the article “Hall Effect Measurements of Surface Conductive Layer of Undoped Diamond Films in NO₂ and NH₃ Atmospheres”, Jpn. J. Appl. Phys. Vol. 38 (1999), p. 3492-3496, R. Sung Gi et al. describe NO₂- and NH₃-induced changes in the electrical surface conductivity in diamond samples with hydrogenated surfaces.

The invention utilizes the effect of the displacement of surface charges through the H-terminated substrate or semiconductor surface, for the first time, for controlled detection of gaseous or vaporous chemical warfare agents.

Other objects, advantages and novel features of the present invention will become apparent from the following detailed description of the invention when considered in conjunction with the accompanying drawings.

### BRIEF DESCRIPTION OF THE DRAWINGS

FIG. 1 is a graph which illustrates the resistance of a hydrogenated diamond at room temperature under different atmospheres;

FIG. 2 is a graph which shows the NH3 response at different temperatures;

FIG. 3 is a graph which shows how the response and decay rate can be enhanced by illumination with ultraviolet light;

FIG. 4 is a graph which illustrates the cleaning of a layer sensitive to various gases by means of ozone;

FIG. 5 is a graph which shows the sensitivity of the hydrogenated surface to NO₂; and

FIG. 6 is a graph which illustrates the enhancement of sensitivity achieved through “dilution” of the hydrogenated surface by partial oxidation.

### DETAILED DESCRIPTION OF THE DRAWINGS

As a preparatory study for production of a warfare agent detector in the form of a gas sensor, an H-terminated surface was produced on a diamond substrate. The monoatomic layer present on the diamond substrate was studied for its sensitivity to various gases at several working temperatures. The layer has been found to be selective to ammonia and NO₂ at room temperature. It is also possible for the response or sensitivity to be increased by partial replacement of the H termination by O termination.

A gas sensor thus produced can be used as a threshold sensor for manufacture in the chemical industry and in the semiconductor industry.

Further possible fields of use are air quality analysis, for example in the transport of foods, in agricultural farms, in biogas plants, in silos, or in the use of coolants.

The studies show that hydrogen (H)-terminated (hydrogenated) semiconductor surfaces are usable for detection of gaseous group III-H, group V-H and group VI-H compounds and other organic gases with these components as bonding partners. These compounds are present in most chemical warfare agents.

Accordingly, the present invention relates to the use of hydrogen (H)-terminated (hydrogenated) semiconductor surfaces as a sensitive layer for detection of chemical warfare agents, in particular the very toxic, gaseous group III-H, group V-H and group VI-H compounds or other organic or inorganic gases with similar functional groups therein.

As the basis for the creation of a warfare agent detector for the highly toxic gaseous group III-H, group V-H and group VI-H compounds, ammonia (NH₃, which is relatively nontoxic in comparison) has been studied in detail as a representative of diborane (B₂H₆), phosphine (Ph₃) and arsine (AsH₃).

Suitable possible substrates to which a hydrogenated sensitive layer can be applied are particularly semiconductor materials, such as diamond, silicon carbide, all group III nitrides (e.g., GaN) and metal oxides (e.g., tin oxide), or else silicon.

A hydrogenated surface is a surface terminated by hydrogen atoms. This termination can be applied in various processes, for example in a hydrogen plasma or in a “hot-wire” hydrogenation. A hydrogenated layer is a monoatomic layer consisting of hydrogen atoms bonded covalently to the lattice constituents of the substrate. The surface is thus coated with a layer of hydrogen atoms. One of several processes suitable for hydrogen termination is hydrogen glow discharge. Further useful processes include silanization and chemical functionalization of surfaces with organic molecules.

Even at relatively low temperatures (e.g., room temperature—RT), these surface hydrogen atoms promote adsorption (in this case, of NH₃ molecules), and form an NH₃—H complex with them. There is a displacement of the surface charge, which can be read out electrically with the aid of suitable measurement technology. For this purpose, a measuring device is used to measure the surface resistance. Measuring devices for measuring ohmic resistance are well known and need not be explained any further here.

Other gases which occur in the environment on a daily basis (e.g., ethanol, hydrocarbons, CO, hydrogen) require higher energies to add onto a hydrogenated surface and to react chemically. Thus, the reducing gases detected at relatively low temperatures (here, lower than 100° C.) are only NH₃ and chemically related substances such as PH₃ and AsH₃. The detection of NH3 by means of a hydrogenated diamond was studied in more detail. The measured results are explained in detail hereinafter with reference to the figures appended.

FIG. 1 shows the resistance behavior of a hydrogenated diamond under different gas atmospheres at room temperature RT. The background atmosphere used was synthetic air SA. In this experiment, gas pulses of different gases in unusually high concentration were introduced at time intervals. One gas pulse was then followed in each case by a purge pulse with synthetic air (SA), which was formed from 20% oxygen and 80% nitrogen. As is evident from the graph of FIG. 1, first 5000 ppm (parts per million) of ethene, then 50 ppm of ethanol, then 500 ppm of carbon monoxide, then 1% hydrogen and finally 5000 ppm of ammonia were introduced. In the course of this, the surface resistance was measured continuously. As is clear from the graph, there was a change in the resistance only when ammonia was introduced.

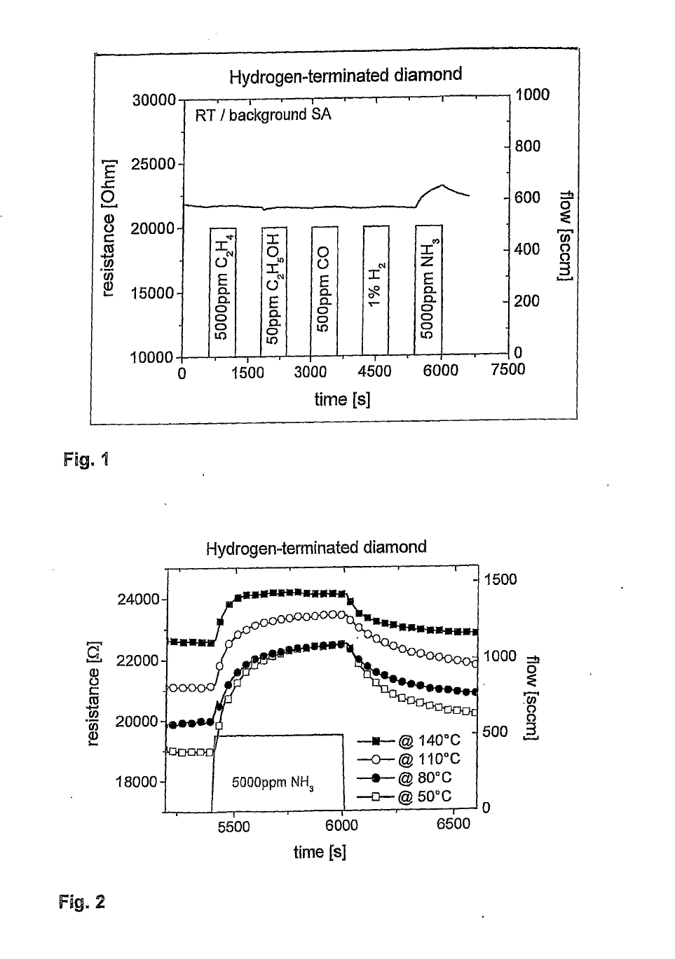

FIG. 2 shows the repetition of the experiment according to FIG. 1, except at successively higher working temperatures of the diamond. Again, only when NH3 was added was an increase in the component resistance observed. When the time period within which NH3 was supplied is considered in more detail, a displacement in the sensor baseline toward a lower base resistance is observed in synthetic air, caused by the higher temperature. With increasing working temperature, however, significantly shorter reaction times for the adsorption and desorption of the NH3 are observed.

FIG. 3 shows the accelerating effect of ultraviolet (UV) light on the response and decay behavior of the diamond sensor. UV illumination is therefore an important alternative to operation of the sensor at elevated temperatures.

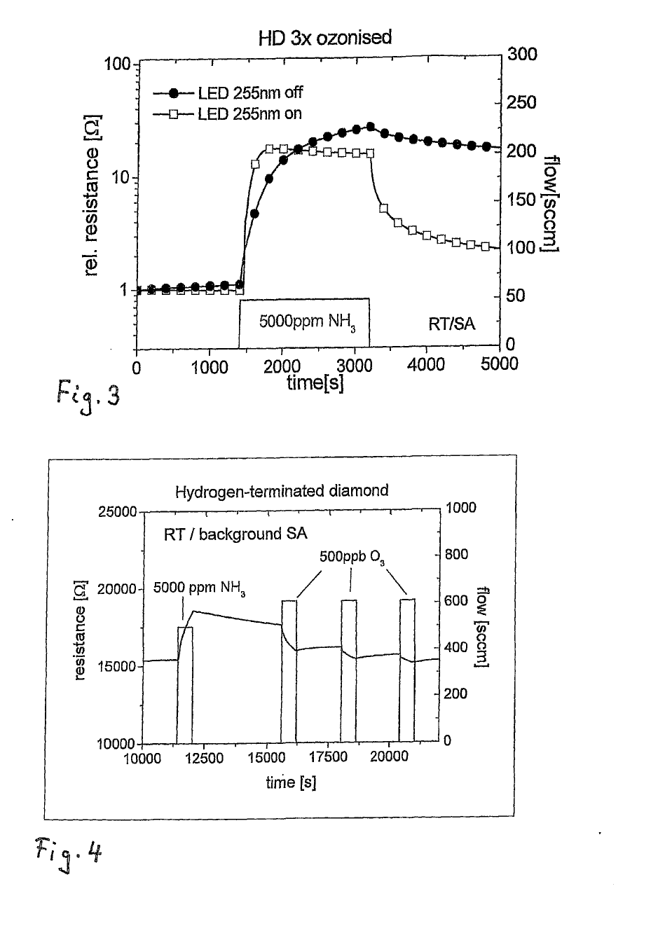

FIG. 4 shows how the desorption of NH₃ is accelerated by addition of ozone. This figure shows how the component resistance at the start of the experiment increases as a result of addition of NH₃. After the gas pulse supplied, a desorption of the ammonia gas from the diamond surface sets in. The resistance of the component decreases slowly. When a very high concentration of ozone is then supplied, the desorption process is accelerated. The resistance of the diamond is reduced drastically during the addition and remains at the lower level for a prolonged period even after the supply of gas. Cleaning of the sensor surface is thus possible with high concentrations of ozone.

### Sensitivity to Oxidizing Gases:

When the sensitivity of a hydrogenated surface to oxidizing gases, for example ozone and NO₂, is considered, it can be stated, as illustrated by FIG. 4, that a cleaning action at the surface sets in when ozone gas is supplied. At extreme ozone concentrations, which typically far exceed environmental concentrations, the hydrogen termination of the surface can be damaged, or replaced partially by oxygen coverage. This latter case is considered further specifically in FIG. 6.

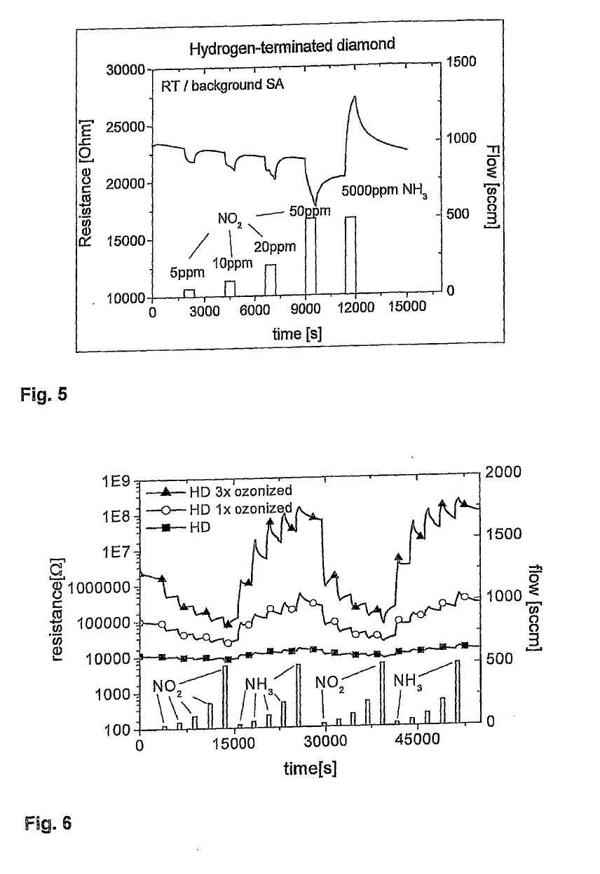

FIG. 5 shows the influence of NO₂. As can be discerned therefrom, NO₂ is the only gas which causes reduction of the component resistance at room temperature. Accordingly, this component can also be used for detection of NO₂, and also of further nitrogen oxides, (NO, N2O).

FIG. 6 shows a repetition of the experiment shown in FIG. 5, in which the gas supply sequence of FIG. 5 was repeated four times in order to test the reproducibility of the results. This experiment was first carried out with a completely hydrogenated diamond and then repeated twice; each repetition was preceded by exposure of the diamond to an extremely aggressive oxygen atmosphere. In these treatments, the number of H-terminated surface bonds is reduced each time. It can be seen that the “dilution” of the H termination and the associated increase in the base resistance cause a significant increase in the gas sensitivity. No occurrence of new gas sensitivities which have not been described to date were observed after these treatments.

Applications for detection of other group III-H compounds or group V-H compounds:

In further experiments which were performed similarly to the experiments explained above, it was possible to demonstrate that a hydrogenated semiconductor surface (especially, the above-discussed sensitive surface) can also be used to detect diborane (B₂H₆) as group III-hydrogen compounds, and also phosphine (PH₃) and arsine (AsH₃) as well as NH3 as further group V-hydrogen compounds.

The studies conducted show that the substrate with the hydrogenated surface safely and reliably detects the chemical warfare agents specified.

The foregoing disclosure has been set forth merely to illustrate the invention and is not intended to be limiting. Since modifications of the disclosed embodiments incorporating the spirit and substance of the invention may occur to persons skilled in the art, the invention should be construed to include everything within the scope of the appended claims and equivalents thereof.

1.-22. (canceled)

23. A method for selectively detecting highly toxic chemical hazardous materials and warfare agents in a gas, said method comprising:
exposing the gas to a substrate with a hydrogen (H)-terminated surface;
wherein, the substrate comprises one of a nonconductor material with available surface conductivity, and a semiconductor material.

24. The method according to claim 23, further comprising reducing density of hydrogen coverage on said surface, by partial oxidation.

25. The method according to claim 23, further comprising measuring displacements of surface charges by means of a measuring device at the hydrogen (H)-terminated surface.

26. The method according to claim 23, wherein the hydrogen (H)-terminated surface comprises a layer of hydrogen atoms applied to the substrate.

27. The method according to claim 26, wherein the layer of hydrogen atoms is a monoatomic layer.

28. The method according to claim 23, wherein the substrate comprises a material selected from the group consisting of diamond, amorphous silicon, silicon carbide, a group III nitride and a metal oxide.

29. The method according to claim 28, wherein the substrate comprises a material selected from the group consisting of GaN or tin oxide or zinc oxide.

30. The method according to claim 23, wherein electrical resistance at the hydrogen (H)-terminated surface is measured by means of a measuring device.

31. The method according to claim 23, wherein detection of said hazardous materials is performed at a temperature below 100° C., especially below approx. 55° C.

32. The method according to claim 31, wherein said detection is performed at room temperature.

33. The method according to claim 23, wherein the sensitive hydrogen (H)-terminated substrate surface is cleaned by purging with a fluid which does not contain said hazardous materials.

34. The method according to claim 33, wherein said fluid is air.

35. The method according to claim 33, wherein the cleaning is performed with addition of an oxidizing fluid.

36. The method according to claim 33, wherein the cleaning is performed with addition of ultraviolet light.

37. The method according to claim 23, wherein said hazardous materials comprise at least one of mustard gas, sulfur mustard gas, organophosphorus warfare agents, nitrogen oxides, B₂H₆, PH₃, AsH₃ or organic warfare agent gases with group III elements, especially Al(CH₃)₃, Ga(CH₃)₃, and In(CH₃)₃.

38. A warfare agent detector for detecting toxic chemical hazardous materials or warfare agents in a gas, said detector comprising:
a substrate which has a hydrogen (H)-terminated substrate surface for exposure to the gas; and
a measuring device for measuring displacements of surface charges at the hydrogen (H)-terminated substrate surface;
wherein, the substrate is made from one of a nonconductor material with surface conductivity, and a semiconductor material.

39. The warfare agent detector as claimed in claim 38, wherein a layer of hydrogen atoms is applied to the substrate to form the hydrogen (H)-terminated substrate surface.

40. The warfare agent detector as claimed in claim 39, wherein the layer of hydrogen atoms is a monoatomic layer.

41. The warfare agent detector as claimed in claim 38, wherein the substrate comprises a material selected from the group consisting of diamond, silicon carbide, a group III nitride and a metal oxide.

42. The warfare agent detector as claimed in claim 41, wherein the substrate comprises a material selected from the group consisting of GaN and tin oxide.

43. The warfare agent detector as claimed in claim 38, wherein the measuring device for measuring the electrical resistance is configured on the hydrogen (H)-terminated substrate surface.

44. A process for producing a warfare agent detector for detecting toxic chemical hazardous materials or warfare agents in a gas, said detector comprising a substrate which has a hydrogen (H)-terminated substrate surface for exposure to the gas; and a measuring device for measuring displacements of surface charges at the hydrogen (H)-terminated substrate surface; wherein, the substrate is made from one of a nonconductor material with surface conductivity, and a semiconductor material, said process comprising:
surface hydrogenating a substrate with the aid of a hydrogen plasma; and
connecting said substrate to a measuring device for readout of a displacement of surface charge.

45. The process as claimed in claim 44, wherein the substrate is a semiconductor substrate comprising a material selected from the group consisting of diamond, silicon carbide, a group III nitride, especially GaN, and a metal oxide.

46. The process according to claim 45, wherein said semiconductor substrate comprise tin oxide.

47. The process as claimed in claim 44, wherein the substrate is a nonconductor with available surface conductivity.
